(19) United States
(12) Patent Application Publication
(10) Pub. No.: US 2016/0213257 A1
(43) Pub. Date: Jul. 28, 2016

(54) PHOTOACOUSTIC APPARATUS

(71) Applicant: CANON KABUSHIKI KAISHA, Ohta-ku, Tokyo (JP)

(72) Inventors: Hiroshi Nishihara, Kawasaki-shi (JP); Ryuichi Nanaumi, Tokyo (JP); Kazuhito Oka, Tokyo (JP); Shuichi Nakamura, Tokyo (JP); Robert A Kruger, Oriental, NC (US)

(21) Appl. No.: 14/916,165

(22) PCT Filed: Sep. 3, 2014
(86) PCT No.: PCT/JP2014/073843

§ 371 Date: Mar. 2, 2016

Related U.S. Application Data
(60) Provisional application No. 61/898,025, filed on Oct. 31, 2013.
(60) Provisional application No. 61/873,542, filed on Sep. 4, 2013.

(51) Int. Cl. A61B 5/00

(52) U.S. Cl. CPC A61B 5/0095

(57) ABSTRACT

A photoacoustic apparatus includes a light source; transducers that receives acoustic waves and outputs electric signals, the acoustic waves being generated when an object is irradiated with light generated from the light source; a support member that supports the transducers such that directivity axes of the transducers gather; a movement region setting unit that sets a movement region of the support member; a moving unit that moves the support member in the movement region such that relative position between the object and the support member changes; and an information acquiring unit that acquires object information based on the electric signals, wherein the light source emits the light when the support member is positioned in the movement region, and wherein the movement region setting unit acquires coordinate information about a surface of the object and determines the movement region based on the coordinate information.

### TECHNICAL FIELD

The present invention relates to a photoacoustic apparatus.

### BACKGROUND ART

Studies of optical imaging apparatuses have been actively conducted in the field of medicine. The optical imaging apparatuses irradiate an object (such as a living body) with light from a light source (such as a laser) and form an image from information about the interior of the object, the information being acquired on the basis of incident light. Photoacoustic imaging (PAI) is one of such optical imaging techniques. In the photoacoustic imaging, an object is irradiated with pulsed light generated from a light source, acoustic waves (typically ultrasonic waves) generated from tissues of the object that absorb energy of the pulsed light that has propagated and that has been diffused in the object are received, and object information is subjected to imaging on the basis of received signals.

That is, by making use of a difference in the rate of absorption of optical energy between a target area (such as a tumor) and other tissues, a search unit receives elastic waves (photoacoustic waves) generated when a test area momentarily expands by absorbing optical energy with which the test area is irradiated. By mathematically analyzing the received signals, it is possible to acquire information about the interior of the object, in particular, a distribution of initial sound pressures, a distribution of optical energy absorption densities, a distribution of absorption coefficients, and the like. These pieces of information can also be used in quantitative measurements of particular materials in the object such as a degree of saturation in blood. In recent years, the photoacoustic imaging has been used to actively conduct preclinical studies in which blood vessels of small animals are imaged, and clinical studies in which the principle of the photoacoustic imaging is applied to the diagnosis of, for example, breast cancer (NPL 1).

PTL 1 describes a photoacoustic apparatus that performs photoacoustic imaging using a search unit in which transducers are disposed at a hemisphere. This search unit is capable of receiving with high sensitivity photoacoustic waves generated in a particular region. Therefore, the resolution of object information for the particular region is increased. PTL 1 also describes that the search unit is used for scanning in a plane, and is then moved in a direction that is perpendicular to the scanning plane to perform scanning in a different plane, and that such scanning operations are performed a plurality of times. According to the scanning that is described in PTL 1, it is possible to acquire object information with high resolution over a wide range.

### CITATION LIST

### Patent Literature

PTL 1 Japanese Patent Laid-Open No. 2012-179348

### Non Patent Literature

NPL 1 “Photoacoustic Tomography: In Vivo Imaging From Organelles to Organs”, Lihong V. Wang Song Hu, Science 335, 1458 (2012))

### SUMMARY OF INVENTION

However, in the scanning that is described in PTL 1, photoacoustic waves may be received even if a region having high sensitivity does not exist in a region for which object information is to be acquired. A received signal that is acquired at this time is a received signal that does not contribute greatly to the acquirement of high-resolution object information for a desired region. That is, in the scanning that is described in PTL 1, the received signal for acquiring the high-resolution object information for the desired region may be acquired with low efficiency.

The present invention provides a photoacoustic apparatus that is capable of efficiently acquiring a received signal for increasing the resolution of object. information for a desired region.

A photoacoustic apparatus includes a light source; a plurality of transducers configured to receive acoustic waves and output electric signals, the acoustic waves being generated when an object is irradiated with light generated from the light source; a support member configured to support the plurality of transducers such that directivity axes of the plurality of transducers gather; a movement region setting unit configured to set a movement region of the support member; a moving unit configured to move the support member in the movement region such that relative position between the object and the support member changes; and an information acquiring unit configured to acquire object information on the basis of the electric signals, wherein the light source emits the light when the support member is positioned in the movement region, and wherein the movement, region setting unit acquires coordinate information about a surface of the object and determines the movement region on the basis of the coordinate information about the surface of the object.

Further features of the present invention will become apparent from the following description of exemplary embodiments with reference to the attached drawings.

### BRIEF DESCRIPTION OF DRAWINGS

FIG. 1 illustrates a structure of a photoacoustic apparatus according to a first embodiment.

FIG. 2 is a graph showing sensitivity characteristics of a transducer.

FIG. 3 illustrates a connection between a computer and peripheral devices thereof.

FIG. 4 is a flowchart showing operations of the photoacoustic apparatus according to the first embodiment.

FIG. 5A illustrates a movement region of a support member.

FIG. 5B illustrates the movement region of the support member.

FIG. 6A illustrates an example in which the support member is linearly moved.

FIG. 6B illustrates the example in which the support member is linearly moved.

FIG. 6C illustrates the example in which the support member is linearly moved.

FIG. 6D illustrates the example in which the support member is linearly moved.

FIG. 6E illustrates the example in which the support member is linearly moved.

FIG. 7A illustrates a modification in which the support member is linearly moved.

FIG. 7B illustrates the modification in which the support member is linearly moved.

FIG. 7C illustrates the modification in which the support member is linearly moved.

FIG. 8 illustrates an example in which the support member is helically moved.

FIG. 9 illustrates an example in which the support member is caused to undergo a three-dimensional spiral movement.

FIG. 10A illustrates an example in which the support member is caused to undergo a plurality of helical movements.

FIG. 10B illustrates the example in which the support member is caused to undergo the plurality of helical movements.

FIG. 11A illustrates an example in which the support member is caused to undergo a plurality of three-dimensional spiral movements.

FIG. 11B illustrates the example in which the support member is caused to undergo the plurality of three-dimensional spiral movements.

FIG. 12A illustrates a modification in which the support member is caused to undergo a plurality of three-dimensional spiral movements.

FIG. 12B illustrates the modification in which the support member is caused to undergo the plurality of three-dimensional spiral movements.

FIG. 13A illustrates a modification in which the support member is caused to undergo a plurality of spiral movements.

FIG. 13B illustrates the modification in which the support member is caused to undergo the plurality of helical movements.

FIG. 14A illustrates a modification in which the support member is caused to undergo a plurality of three-dimensional spiral movements.

FIG. 14B illustrates the modification in which the support member is caused to undergo the plurality of three-dimensional spiral movements.

FIG. 15A illustrates an example in which the support member is caused to undergo a plurality of two-dimensional spiral movements.

FIG. 15B illustrates the example in which the support member is caused to undergo the plurality of two-dimensional spiral movements.

FIG. 15C illustrates the example in which the support member is caused to undergo the plurality of two-dimensional spiral movements.

FIG. 15D illustrates the example in which the support member is caused to undergo the plurality of two-dimensional spiral movements.

FIG. 15E illustrates the example in which the support member is caused to undergo the plurality of two-dimensional spiral movements.

FIG. 16 illustrates a structure of a photoacoustic apparatus according to a fifth embodiment.

FIG. 17 illustrates refraction at a shape maintaining unit.

### DESCRIPTION OF EMBODIMENTS

### First Embodiment

A photoacoustic apparatus according to the present embodiment is an apparatus that acquires object information on the basis of received signals of photoacoustic waves.

The photoacoustic apparatus according to the present invention includes a light source that emits light for generating photoacoustic waves. The photoacoustic apparatus according to the present embodiment also includes a support member that supports a plurality of transducers so as to gather directivity axes such that photoacoustic waves generated at a particular region by application of light can be received with high sensitivity. The photoacoustic apparatus according to the present embodiment also includes a moving unit that moves the support member with respect to an object. The photoacoustic apparatus according to the present embodiment also includes a movement region setting unit that acquires coordinate information about a surface of the object and sets a movement region of the support member on the basis of the coordinate information about the surface of the object. That is, the movement region setting unit according to the present embodiment is capable of changing the movement region of the support member. The light source according to the present embodiment emits light when the support member is positioned in the movement region.

The photoacoustic apparatus according to the present embodiment is capable of preferentially receiving with high sensitivity photoacoustic waves that are generated from the interior of the object. That is, the photoacoustic apparatus according to the present embodiment is capable of efficiently acquiring received signals for increasing the resolution of the object information about the interior of the object.

The term “measure” in the description refers to application of light and reception of photoacoustic waves generated by the application of light. The term “measurement position” refers to the position of a search unit when light is applied, that is, the position of the support member. The term “measurement timing” refers to a timing when an object is irradiated with light.

The embodiment according to the present invention is hereunder described with reference to the drawings. However, for example, the dimensions, materials, shapes, and relative arrangements of structural components described below are to be changed as required due to various conditions and structures of the apparatus to which the present invention is applied. The scope of the invention is not limited to the description below.

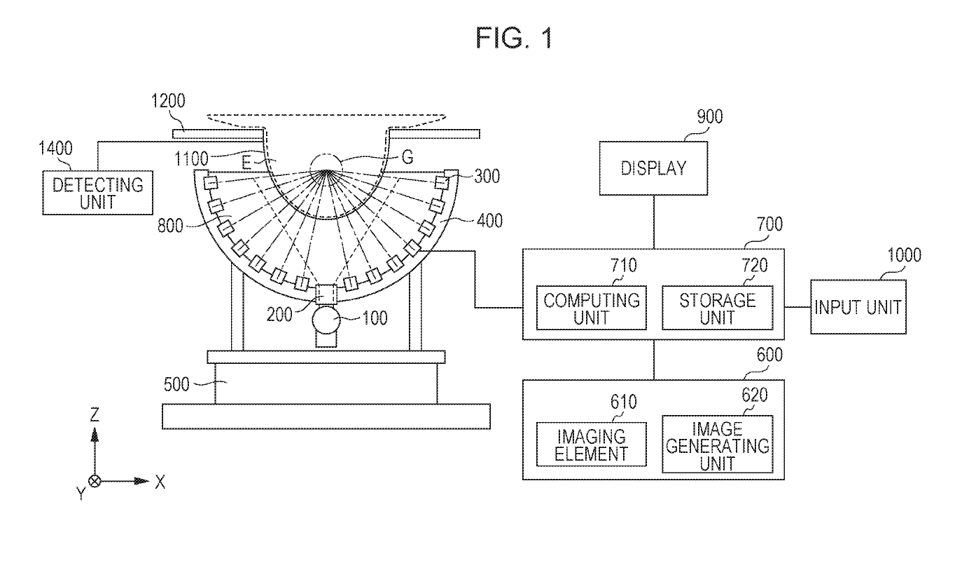

The photoacoustic apparatus according to the first embodiment is described below. FIG. 1 is a schematic view of a structure of the photoacoustic apparatus according to the present embodiment. The photoacoustic apparatus according to the present embodiment sets a movement, region of the support member on the basis of coordinate information about a surface of an object.

The photoacoustic apparatus shown in FIG. 1 is an apparatus that acquires information (object information), such as optical characteristics of an object E, on the basis of received signals of photoacoustic waves generated on the basis of a photoacoustic effect.

Examples of the object information that can be acquired by the photoacoustic apparatus according to the present embodiment include a distribution of initial sound pressures of photoacoustic waves, a distribution of optical energy absorption densities, a distribution of absorption coefficients, and a distribution of concentrations of materials that form the object. The concentrations of materials include, for example, a degree of oxygen saturation, an oxyhemoglobin concentration, a deoxyhemoglobin concentration, and a total hemoglobin concentration. The total hemoglobin concentration is the sum of the concentrations of oxyhemoglobin and deoxyhemoglobin.

### Basic Structure

The photoacoustic apparatus according to the present embodiment includes a light source 100, an optical system 200, a plurality of transducers 300, a support member 400, a scanner 500, an imaging device 600, a computer 700, a display 900, an input unit 1000, and a shape maintaining unit 1100.

Each structural component of the photoacoustic apparatus and a structure used in measurement are hereunder described.

### Object

The object E is an object to be measured. Specific examples thereof include a living body, such as a breast, and a phantom in which acoustic characteristics and optical characteristics of a living body are simulated in, for example, adjusting a device. The term “acoustic characteristics” specifically refers to a propagation speed and an attenuation factor of acoustic waves. The term “optical characteristics” specifically refers to a light absorption coefficient and a light scattering coefficient. It is necessary that a light absorber having a large light absorption coefficient exist in the interior of the object. In a living body, for example, hemoglobin, water, melanin, collagen, and fat become the light absorber. In a phantom, a material in which optical characteristics are simulated is, as a light absorber, sealed in the interior. For convenience, the object E is indicated by dotted lines in FIG. 1.

### Light Source

The light source 100 is a device that generates pulsed light. In order to provide a large output, the light source is desirably a laser. However, a light emitting diode or the like may be used. In order to effectively generate photoacoustic waves, it is necessary to irradiate the object with light for a sufficiently short time in accordance with the heat characteristics of the object. When the object is a living body, it is desirable that the pulse width of the pulsed light, that is generated from the light source 100 be less than or equal to a few tens of nanoseconds. The wavelength of the pulsed light is in a near-infrared region, which is called a window of a living body, and is desirably on the order of 700 nm to 1200 nm. Light in this region can reach a relatively deep portion of a living body, so that information about the deep portion can be acquired. If measurement is limited to that of a surface portion of a living body, light from the visible light region to the near-infrared region of from approximately 500 to 700 nm may be used. Further, it is desirable that the wavelength of the pulsed light have a large absorption coefficient with respect to an object to be observed.

### Optical System

The optical system 200 is a device that guides the pulsed light generated by the light source 100 to the object E. More specifically, the optical system 200 includes optical devices such as a lens, a mirror, a prism, an optical fiber, and a diffusing plate. When the light is guided, using these optical device components, the shape and optical density may be changed so that a desired light distribution is set. Examples of optical device components are not limited to those mentioned here. As long as such functions are satisfied, any optical device components may be used. The optical system 200 according to the present embodiment is formed so as to illuminate a region at a center of curvature of a hemisphere.

The intensity of light allowing irradiation of tissues of a living body is such that maximum permissible exposure (MPE) is prescribed by safety standards indicated below (IEC 60825-1: Safety of laser products, JIS C 6802: Safety standards of laser products, FDA: 21CFR Part 1040. 10, ANSI Z136.1: Laser Safety Standards, etc.). The maximum permissible exposure prescribes the intensity of light that can be applied per unit area. Therefore, by applying light all at once to a surface of the object E using a wide area, a large amount of light can be guided to the object E. Therefore, it is possible to receive photoacoustic waves with a high SN ratio. Consequently, it is desirable that the area be increased to a certain area by condensing the light with a lens, as indicated by a broken line shown in FIG. 1.

### Transducer

Each transducer 300 is an element that receives photoacoustic waves and converts them into electric signals. It is desirable that the frequency bandwidth be wide and the receiving sensitivity be high with respect to photoacoustic waves from the object E.

Examples of materials of transducers 300 that may be used include piezoelectric ceramic materials as typified by lead zirconate titanate (PZT), and piezoelectric polymer film materials as typified by polyvinylidene fluoride (PVDF). Elements other than piezoelectric elements may be used. For example, capacitive elements such as cMUT (capacitive micro machined ultrasonic transducers) and transducers using Fabry-Perot interferometers may be used.

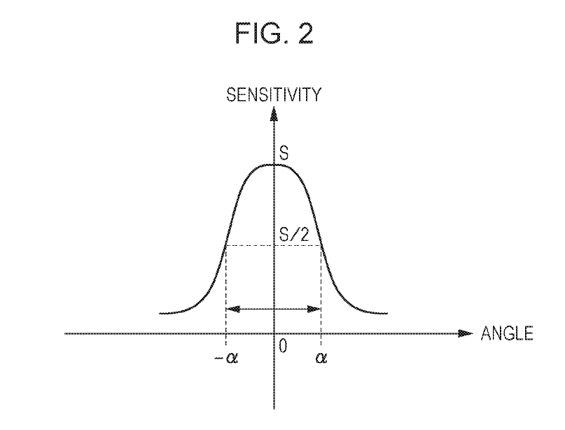

FIG. 2 is a graph showing receiving sensitivity characteristics of a transducer 300. The receiving sensitivity characteristics shown in FIG. 2 correspond to those based an incidence angle between a direction that is normal to a receiving surface of the transducer 300 and a direction of incidence of photoacoustic waves. In the example shown in FIG. 2, the receiving sensitivity when the light is incident from the direction that is normal to the receiving surface is highest. The receiving sensitivity becomes lower as the incidence angle is increased. Each transducer 300 according to the present embodiment is assumed as having a circular planar receiving surface.

An incidence angle when the receiving sensitivity becomes half S/2 of a maximum value S of the receiving sensitivity is α. In the present embodiment, a region of the receiving surface of a transducer 300 upon which photoacoustic waves are incident at an angle less than or equal to the incidence angle α is defined as a receiving region capable of receiving photoacoustic waves with high sensitivity.

In FIG. 1, a highest receiving sensitivity direction of each transducer 300 is indicated by alternate long and short dashed lines. An axis along the highest receiving sensitivity direction of each transducer 300 is called a directivity axis.

### Support Member

The support member 400 is a container having a substantially hemispherical shape formed by cutting a sphere in half. The plurality of transducers 300 are arranged at a surface at an inner side of the hemispherical support member 400. The optical system 200 is disposed at a bottom portion (pole) of the support member 400. The inner side of the support member 400 is filled with an acoustic matching material 800 (described later).

It is desirable that the support member 400 be formed of, for example, a metallic material having a high mechanical strength for supporting these members.

The plurality of transducers 300, provided at the support member 400, are disposed at a hemispherical surface so that receiving directions of the plurality of transducers 300 differ from each other and are towards the center of curvature of the hemisphere. FIG. 1 is a sectional view in which the hemispherical support member 400 is sectioned at a center axis, with alternate long and short dashed lines that converge in a region of a portion of the interior of the object E indicating the receiving directions of the transducers 300.

By causing the directivity axes of the plurality of transducers 300 to gather in this way, compared to the case in which the directivity axes of the plurality of transducers 300 are parallel to each other, it is possible to receive with higher sensitivity photoacoustic waves generated at a particular region (near the center of curvature of the support member 400). In the present embodiment, this particular region is called a high sensitivity region.

When such plurality of transducers 300 are arranged, object information that is acquired using received signals using a method described below is such that the resolution at the center of curvature of the hemisphere is high and the resolution is reduced with increasing distance from the center. The high sensitivity region in the present embodiment refers to a region from a point where the resolution is highest to a point where the resolution becomes half of the highest resolution, and corresponds to a region G that is surrounded by alternate long and two short dashes lines in FIG. 1.

For example, the high sensitivity region G can be set as a substantially spherical region having a radius r indicated in Formula (1) with a point where a highest resolution R_{H} is obtained being the center:

$$[ Math . 2 ]$$

$$r = \frac{{r}_{0}}{{φ}_{d}} \sqrt{{R}^{2} - {R}_{H}^{2}} \qquad ( 1 )$$

where R is a lower limit resolution of the high sensitivity region G, R_{H} is the highest resolution, r₀ is the radius of the hemispherical support member 400, and φ_{d} is the diameter of a transducer. For example, the lower limit resolution is a resolution that is half of the highest resolution. When the support member 400 has a hemispherical shape, the center of curvature of the support member 400 is typically where the resolution is highest.

The case in which the high sensitivity region G is substantially spherical with the point of center of curvature of the support member 400 being the center is considered. In this case, the range of high sensitivity region G at each measurement timing can be estimated from the position of the support member 400, that is, the position of the center of curvature and Formula 1.

As long as a desired high sensitivity region can be formed, the plurality of transducers 300 may be arranged in any way. The highest sensitivity directions of the plurality of transducers 300 need not intersect, at one point.

In order to receive with high sensitivity photoacoustic waves generated at a particular region, all that is required is for the highest receiving sensitivity directions of at least some of the plurality of transducers 300 that are supported by the support member 400 be toward a particular region. That is, all that is required is for the plurality of transducers 300 be arranged at the support member 400 so that at least some of the plurality of transducers 300 are capable of receiving with high sensitivity photoacoustic waves that are generated at a high sensitivity region.

All that is required is for the plurality of transducers 300 be arranged at the support member 400 so that the directivity axes of the plurality of transducers 300 are gathered compared to the case in which the highest receiving sensitivity directions of the plurality of transducers 300 are parallel to each other.

The plurality of transducers 300 may be arranged so that the receiving surfaces of the plurality of transducers 300 are placed along the support member 400. Here, the shape of the support member 400 is not limited to a hemispherical shape such as that in the present embodiment. As long as the plurality of transducers 300 are arranged as described above, the support member 400 may have a shape including any curved surface. The term “curved surface” in the present embodiment also refers to a curved surface other than a spherical surface. That is, the term “curved surface” in the present embodiment also refers to an uneven surface that is uneven to the extent that allows it to be considered as a curved surface and a surface of an ellipsoid (which is a three-dimensional analog of an ellipse and has a two-dimensional curved surface) that is elliptical to the extent that allows it to be considered as a curved surface. Further, the term “curved surface” in the present embodiment refers to a surface that is formed by connecting a plurality of planar surfaces. The term “receiving surface” in the present embodiment refers to a surface that is provided in a direction that is normal to the highest receiving sensitivity direction.

It is desirable that the plurality of transducers 300 be arranged at the support member 400 so that the receiving surfaces of the plurality of transducers 300 face the inner side of the support member 400. In the present embodiment, the side of the center of curvature of the support member 400 corresponds to the inner side of the support member 400.

It is desirable that the plurality of transducers 300 be arranged so that a high sensitivity region that is determined by the arrangement of the plurality of transducers 300 be formed at a position where the object E is assumed to be positioned. When the shape maintaining unit 1100 that maintains the shape of the object E is provided as in the present embodiment, the plurality of transducers 300 are arranged so as to form a high sensitivity region near the shape maintaining unit 1100.

### Scanner

The scanner 500, serving as a moving unit, is a device that changes the position of the support member 400 relative to the object E by moving the position of the support member 400 in directions X, Y, and Z in FIG. 1. Therefore, the scanner 500 includes a guide mechanism for performing guiding in the directions X, Y, and Z (not shown), a driving mechanism for performing driving in the directions X, Y, and Z, and a position sensor that receives the position of the support member 400 in the directions X, Y, and Z. As shown in FIG. 1, the support member 400 is placed above the scanner 500. Therefore, the guide mechanism is desirably, for example, a linear guide that is capable of withstanding a large load. Examples of the driving mechanism that may be used include a lead screw mechanism, a link mechanism, a gear mechanism, and a hydraulic mechanism. Driving force may be generated by, for example, a motor. The position sensor may be, for example, a potentiometer using, for example, an encoder or a variable resistor.

In the present invention, since all that is required is for the relative position between the object E and the support member 400 be changed, it is possible to fix the support member 400 and move the object E. When the object E is moved, a structure that moves the object E by moving a support unit (not shown) that supports the object E may be considered. Further, it is possible to move both the object E and the support member 400.

It is desirable for the movement to be continuous. However, the movement may be repeated in certain steps. Although it is desirable for the scanner 500 to be an electric stage, it may be a manual stage. However, the scanner 500 is not limited to those mentioned above. As long as at least one of the object E and the support member 400 is movable, any structure may be used.

### Imaging Device

The imaging device 600 generates image data of the object E and outputs the generated image data to the computer 700. The imaging device 600 includes an imaging element 610 and an image generating unit 620. The image generating unit 620 generates the image data of the object E by analyzing a signal output from the imaging element 610, and causes the generated image data to be stored in a storage unit 720 in the computer 700.

For example, an optical imaging element, such as a charge-coupled device (CCD) sensor or a complementary metal-oxide semiconductor (CMOS) sensor, may be used as the imaging element 610. For example, a transducer that transmits and receives photoacoustic waves, such as a piezoelement or a capacitive micro-machined ultrasonic transducer (CMUT), may be used as the imaging element 610. Some of the plurality of transducers 300 may be used for the imaging element 610. As long as the image generating unit 620 is capable of generating an image of the object on the basis of a signal output from the imaging element 610, any element may be used for the imaging element.

The image generating unit 620 may include an element, such as a central processing unit (CPU), a graphics processing unit (GPU), or an analog-to-digital (A/C) converter; or a circuit, such as a field programmable gate array (FPGA) or an application specific integrated circuit (ASIC). The computer 700 may also function as the image generating unit 620. That is, a computing unit in the computer 700 may be used as the image generating unit 620.

The imaging device 600 may be provided separately from the photoacoustic apparatus.

### Computer

The computer 700 includes the computing unit 710 and the storage unit 720.

The computing unit 710 typically includes an element, such as a central processing unit (CPU), a graphics processing unit (CPU), or an analog-to-digital (A/C) converter; or a circuit, such as a field programmable gate array (FPGA) or an application specific integrated circuit (ASIC). The computing unit may be formed not only by a single element or circuit, but also by a plurality of elements or circuits. Also, each processing operation performed by the computer 700 may be performed by any of the elements or circuits.

The storage unit 720 typically includes a storage medium, such as a read-only memory (ROM), a random-access memory (RAM), or a hard disk. The storage unit may be formed not only by a single storage medium, but also by a plurality of storage media.

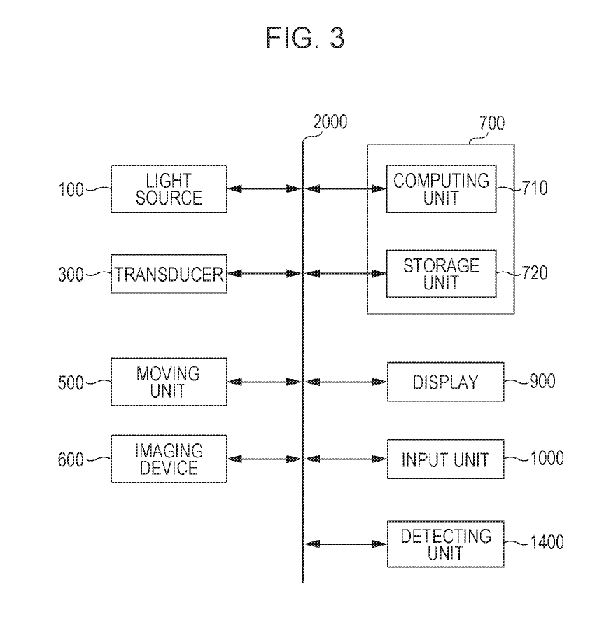

The computing unit 710 is capable of processing electric signals output from the plurality of transducers 300. As shown in FIG. 3, the computing unit 710, serving as a controlling unit, is capable of controlling the operation of each structural component of the photoacoustic apparatus via a bus 2000.

It is desirable that the computer 700 be configured to perform pipeline processing of a plurality of signals at the same time. This can reduce the time necessary to acquire object information.

Each processing operation performed by the computer 700 can be stored in the storage unit 720 as a program to be executed by the computing unit 710. Note that the storage unit 720 where the program is stored is a non-transitory recording medium.

### Acoustic Matching Material

The acoustic matching material 800 fills up a space between the object E and the transducers 300, and acoustically couples the object E and the transducers 300. In the present embodiment, the acoustic matching material 800 is disposed between the shape maintaining unit 1100 and the object E.

The acoustic matching material 800 may also be provided between the transducers 300 and the shape maintaining unit 1100. Different acoustic matching materials may be provided between the transducers 300 and the shape maintaining unit 1100 and between the shape maintaining unit 1100 and the object E.

It is desirable that the acoustic matching material 800 be a material in which photoacoustic waves are less likely to be attenuated in the interior of the acoustic matching material 800. It is desirable that the acoustic matching material 800 be a material whose acoustic impedance is close to those of the object E and the transducers 300. In addition, it is desirable that the acoustic matching material 800 be a material having an acoustic impedance that is intermediate between those of the object E and the transducers 300. Further, it is desirable that the acoustic matching material 800 be a material that transmits pulsed light generated by the light source 100 therethrough. Still further, it is desirable that the acoustic matching material 800 be a liquid. More specifically, the acoustic matching material 800 may be, for example, water, castor oil, or gel.

The acoustic matching material 800 may be provided separately from the photoacoustic apparatus according to the present invention.

### Display

Using, for example, distribution image and numerical data, the display 900, serving as a display unit, displays object information that is output from the computer 700. Although a liquid crystal display or the like is typically used as the display 900, a plasma display, an organic electro-luminescent (EL) display, or a field emission display (FED) may also be used. The display 900 may be provided separately from the photoacoustic apparatus.

### Input Unit

The input unit 1000 is a member configured to allow desired information to be specified for inputting the desired information to the computer 700 by a user. Examples of the input unit 1000 include a keyboard, a mouse, a touch panel, a dial, and a button. When a touch panel is used as the input unit 1000, the touch panel may be one in which the display 900 also functions as the input unit 1000. The input unit 1000 may be provided separately from the photoacoustic apparatus according to the present embodiment.

### Shape Maintaining Unit

The shape maintaining unit 1100 is a member for maintaining the shape of the object E in a certain shape. The shape maintaining unit 1100 is mounted on a mount unit 1200. When a plurality of shape maintaining units for maintaining a plurality of shapes of the object E are used, it is desirable that the mount unit 1200 be configured to allow the plurality of shape maintaining units to be mounted thereon or be removed.

When light is applied to the object E via the shape maintaining unit 1100, it is desirable that the shape maintaining unit 1100 be transparent to the applied light. For example, the shape maintaining unit 1100 may be formed of polymethylpentene or polyethylene terephthalate.

When the object E is a breast, in order to maintain the shape of the breast in a certain shape by reducing deformation thereof, it is desirable that the shape maintaining unit 1100 have a shape formed by sectioning a sphere by a certain section. It is possible to form the shape of the shape maintaining unit 1100 as appropriate in accordance with the volume of the object and a maintained desired shape. It is desirable that the shape maintaining unit 1100 fit the external shape of the object and that the shape of the object E have substantially the same shape as the shape maintaining unit 1100. The photoacoustic apparatus may perform measurement without using the shape maintaining unit 1100.

### Operation of Photoacoustic Apparatus

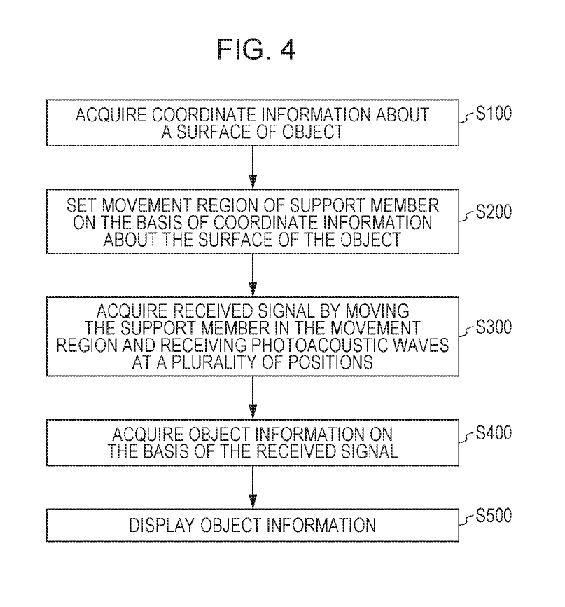

Next, using the flowchart in FIG. 4, a method for efficiently receiving photoacoustic waves generated in an object on the basis of coordinate information about a surface of the object is described.

S100: Step for Acquiring Coordinate Information about a Surface of an Object

First, an object E is inserted into the shape maintaining unit 1100, and a space between the support member 400 and the shape maintaining unit 1100 and a space between the shape maintaining unit 1100 and the object. E are filled with acoustic matching materials 800.

Next, the computing unit 710 acquires coordinate information about a surface of the object E. The method for acquiring the coordinate information about the surface of the object E using the computing unit 710 is hereunder described.

First, the computing unit 710 reads out from the storage unit 720 image data of the object E acquired by the imaging device 600. Next, on the basis of the image data of the object E, the computing unit 710 computes the coordinate information about the surface of the object E. For example, it is possible to compute the coordinate information about the surface of the object E using a three-dimensional measurement technique, such as a stereo method, on the basis of a plurality of pieces of image data. It is possible for the computing unit 710 to acquire the coordinate information about the surface of the object on the basis of information about position coordinates of the surface of the object. E.

Alternatively, previously known coordinate information about a surface of the shape maintaining unit 1100 may be stored in the storage unit 720. The computing unit 710 can acquire the coordinate information about the surface of the object E by reading the coordinate information about the surface of the shape maintaining unit 1100 from the storage unit 720. It is possible to provide a detecting unit 1400 that detects the type of shape maintaining unit mounted on the mount unit 1200 and outputs information about the type of shape maintaining unit to the computer 700. The computing unit 710 can receive the information about the type of shape maintaining unit output from the detecting unit 1400, and acquire, as the coordinate information about the surface of the object, the coordinate information about the surface of the shape maintaining unit corresponding to the received information about the type of shape maintaining unit. For example, the detecting unit 1400 may be a reader that reads an ID chip mounted on the shape maintaining unit and indicating the type of shape maintaining unit mounted. This makes it possible to acquire the coordinate information about the surface of the object without performing calculations.

Alternatively, a user may use the input, unit 1000 to input information on the type of shape maintaining unit that is used, as a result of which the input unit 1000 outputs input information to the computer 700. The computing unit 710 can receive the information about the type of shape maintaining unit output from the input unit 1000, and acquire, as the coordinate information about the surface of the object, coordinate information about the surface of the shape maintaining unit corresponding to the received information about the type of shape maintaining unit. This makes it possible to acquire the coordinate information about the surface of the object without performing calculations.

When it is assumed that the type of shape maintaining unit does not change, so that it is not assumed that the size of the shape maintaining unit changes in terms of the specification, it is possible for the coordinate information about the surface of the object that is used by the computing unit 710 to be fixed.

It is possible to acquire the coordinate information about the surface of the object E using a contact probe.

When the photoacoustic apparatus is to perform a plurality of measurements, the coordinate information about the surface of the object acquired in this step may be used in a later measurement. In addition, when the photoacoustic apparatus is to perform a plurality of measurements, it is possible to perform this step at any timing, such as at each measurement or after every few measurements.

Even if the shape of the object has changed between measurements as a result of performing this step at each measurement, it is possible to perform a later step on the basis of precise coordinate information about the surface of the object each time the shape changes.

S200: Step for Setting a Movement Region of the Support Member on the Basis of Coordinate Information about a Surface of an Object

Next, the computing unit 710, serving as a movement region setting unit, sets a movement region of the support member 400 on the basis of the coordinate information about the surface of the object E acquired in S100.

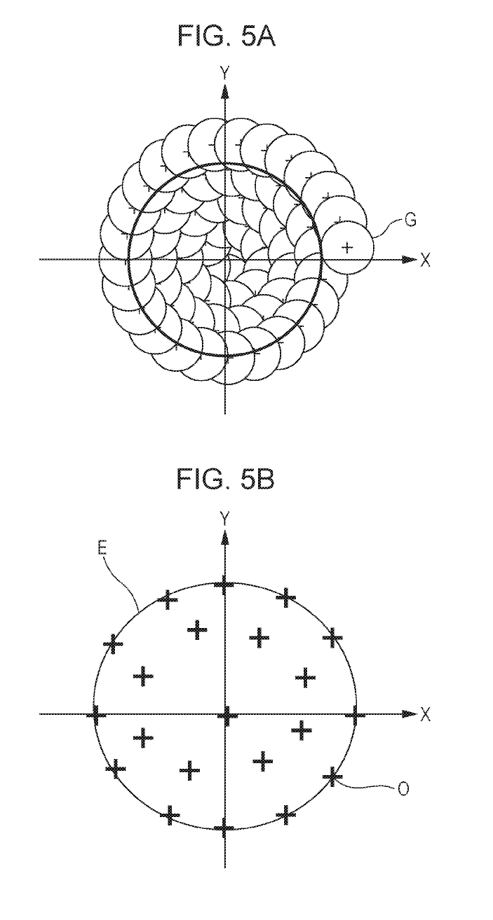

At this time, the computing unit 710 sets a movement region in the directions X, Y, and Z of the support member 400 on the basis of the coordinate information about the surface of the object E so that a high sensitivity region G is formed at an inner side of the object E as shown in FIG. 5A. The position and the size of the high sensitivity region G is determined by the arrangement of the plurality of transducers 300. On the basis of the coordinate information about the surface of the object E and information about the arrangement of the plurality of transducers 300 at the support member 400, the computing unit 710 sets the movement region so as to perform measurement when the high sensitivity region G is formed at the inner side of the object E. Information about the size and position of the high sensitivity region G that is determined from the arrangement of the plurality of transducers 300 may be previously stored in the storage unit 720. In this case, the computing unit 710 sets the movement region on the basis of the information about the size and position of the high sensitivity region G read out from the storage unit 720 and the coordinate information about the surface of the object E.

As shown in FIG. 5B, it is desirable to set the movement region of the support member 400 so as to perform measurement when a center O of the high sensitivity region G at each measurement position indicated by a cross (+) is formed at the inner side of the object E. That is, in the present embodiment, it is desirable that the movement region be set so as to perform measurement when the object 3 exists at the center of curvature of the hemispherical support member 400 at the measurement positions.

Further, it is desirable that the movement region be set so as to perform measurement when the center of the high sensitivity region G corresponding to an outermost periphery of the movement region matches an outer edge of the object H as shown in FIG. 5B.

By setting the movement region such as that described above, it is possible to receive with high sensitivity photoacoustic waves generated in a wide range within the object E even if the movement region is small. As a result, the acquired object information about the interior of the object has high resolution in a wide range. Since the movement region is small, it is possible to reduce an entire measurement time.

The computing unit. 710, serving as a path setting unit, is capable of setting as appropriate a movement path of the support member 400 in the movement region.

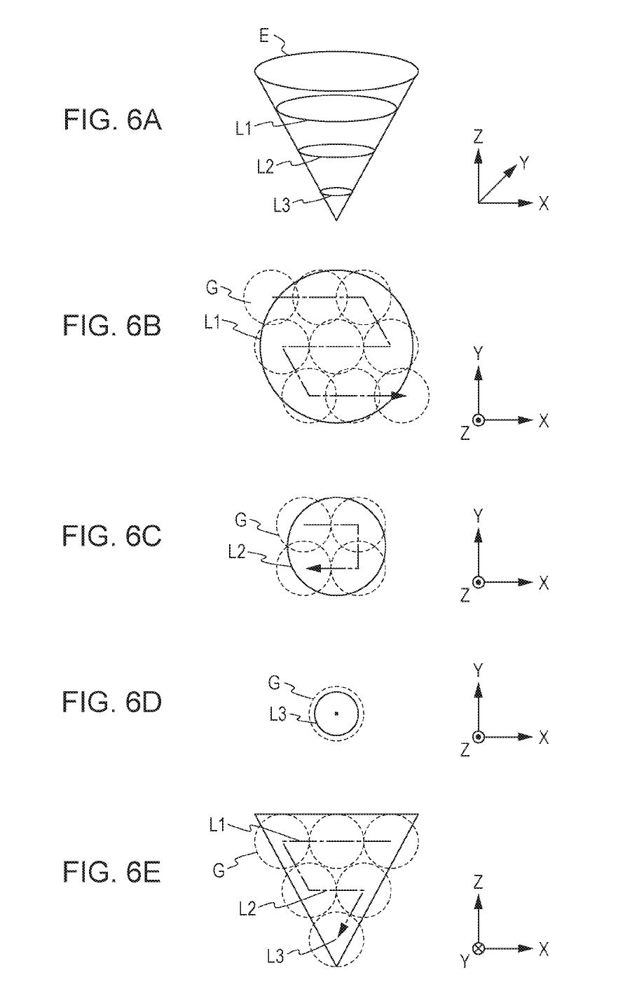

Here, an example in which the support member 400 is caused to undergo linear movement and a change of direction in a conical movement region at a conical object such as that shown in FIG. 6A is described. The cross sections of a cone differ in the height direction (direction Z). When each section differs as in the cone, as shown in FIG. 6A, it is desirable to set the movement region of the support member 400 by dividing the object into a plurality of layers considering the size of the high sensitivity region G. In the embodiment, the movement region is set by dividing the conical object in three layers L1, L2, and L3. FIGS. 6B to 6B illustrate, in an X-Y plane, a path (alternate long and short dashed lines) of the center of the high sensitivity region G resulting from the movement of the support member 400 at the layers L1 to L3 and the high sensitivity region G (dotted circles) at each measurement position. FIG. 6E illustrates, in an X-Z plane, a path of the center of the high sensitivity region G and the high sensitivity region G at each measurement position.

On the basis of the coordinate information about the surface of the object and the size and position of the high sensitivity region G, the computing unit 710 computes the positions of change of direction and the movement path shown in FIGS. 6B, 6C, 6D, and 6E, and sets the movement region in which the support member 400 moves suitable for the conical object.

The computing unit 710 is capable of setting as appropriate measurement positions of photoacoustic waves within the set movement region. It is possible to set the measurement positions at certain intervals within the set movement, region. That is, the computing unit 710 is capable of controlling driving of the scanner 500 and the light source 100 so that the measurement positions are provided at certain intervals.

Further, it is desirable that the driving of the scanner 500 and the light source 100 be controlled so that the high sensitivity regions G at the measurement positions overlap. That is, since, in the present embodiment, the high sensitivity regions G are spherical, it is desirable that pulsed light be applied at least once until the support member 400 moves by a distance that is equal to the radius of the high sensitivity regions G. This means that a received signal is acquired at least once while the support member 400 moves through a distance that is equivalent to the radius of the high sensitivity regions G.

The smaller the distance through which the support member 400 moves until a next application of light from a certain application of light, the more uniform is the resolution. However, the smaller the movement distance (that is, the lower the movement speed), the longer an overall measurement time. Therefore, it is desirable to set, as appropriate, the movement speed and the time interval between acquirements of received signals considering the desired resolution and measurement times.

### S300: Step for Acquiring Received Signal by Moving the Support Member in Movement Region and Receiving Photoacoustic Waves at a Plurality of Positions in the Movement Region

The scanner 500 moves the support member 400 to a first measurement position where a measurement is started in the movement region that has been set in S200. At this time, the scanner 500 successively transmits coordinate information about the support member 400 to the computer 700.

When, on the basis of the coordinate information about the support member 400 transmitted from the scanner 500, the computing unit. 710 determines that the support member 400 is at the first measurement position, the computing unit 710 outputs a control signal so as to cause the light source 100 to generate light. The light is guided to the optical system 200, and is applied to the object E via the acoustic matching material 800. The light applied to the object E is absorbed by the interior of the object E, so that photoacoustic waves are generated. At this time, coordinate information about the support member 400 when the light is applied is transmitted from the scanner 500 to the computer 700, and this is stored in the storage unit 720 as coordinate information about the support member 400 at the first measurement position.

The plurality of transducers 300 receive the photoacoustic waves generated in the interior of the object E and propagated through the interior of the acoustic matching material 800, and convert them into electric signals serving as received signals.

The electric signals output from the transducers 300 are transmitted to the computer 700, are associated with the first measurement position information, and are stored in the storage unit 720 as electric signals for the first measurement position.

Next, the scanner 500 moves the support member 400 to a second measurement position differing from the first measurement position in the movement region that has been set in S200. Then, when the support member 400 is at the second measurement position, the operations that are the same as the measurements performed at the first measurement position are performed, so that electric signals for the second measurement position are acquired. Thereafter, by performing the operations that are the same as those described above, electric signals are acquired for all the other measurement positions that have been set in the movement region that has been set in S200.

In this step, photoacoustic waves are generated when the high sensitivity regions G overlap the object E at the measurement positions. Therefore, a received signal acquired at either of these measurement positions is also a received signal that is output as a result of reception by the plurality of transducers 300 of the photoacoustic waves generated in the interior of the object E with high sensitivity. Since the movement region of the support member 400 is set so as not to generate and receive photoacoustic waves when a high sensitivity region G does not exist at the object E, a received signal that contributes to acquirement of the object information about the interior of the object E can be efficiently acquired.

S400: Step for Acquiring Object Information Based on Received Signals

The computing unit 710, serving as an information acquiring unit, acquires the object information by processing, on the basis of an image reconstruction algorithm, the received signals acquired in S300.

For example, as the image reconstruction algorithm for acquiring the object information, reverse projection methods including a time domain method and a Fourier domain method ordinarily used in tomographic technology are used. When it is possible to have a long reconstruction time, it is possible to use an image reconstruction method such as an inverse problem analysis based on repeated operations.

As mentioned above, the received signals acquired in S300 are received signals that are acquired by receiving with high sensitivity the photoacoustic waves generated in the interior of the object E. Therefore, it is possible to precisely acquire the object information about the interior of the object E in this step. That is, the resolution and quantitativity of the object information about the interior of the object E acquired in this step are high.

Although in FIGS. 6A to 6E, a shape in which each cross section differs in the direction Z is exemplified, it is also possible to apply the present embodiment to the case in which the cross section does not change in the direction Z as in a cylinder or a prism. In this case, the computing unit 710 may set the same movement region of the support member 400 for each cross section.

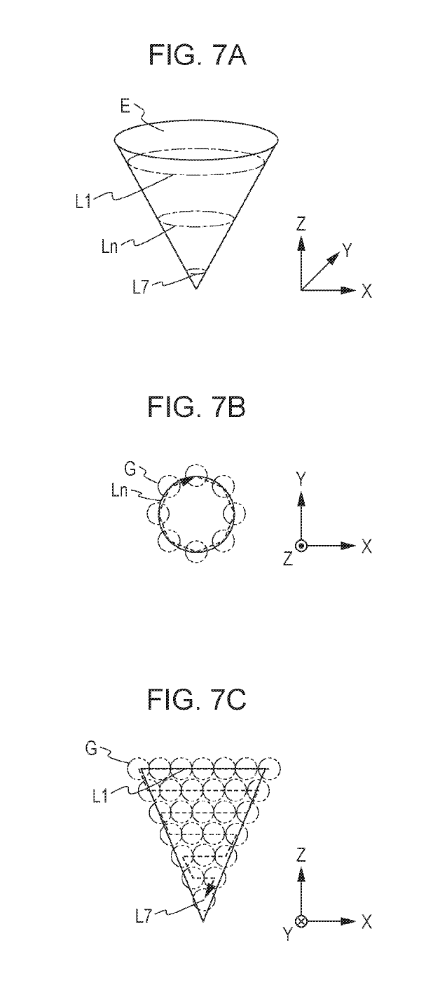

As shown in FIGS. 7A to 7C, it is possible to set a movement region and a movement path of the support member 400 so that the center of the high sensitivity region G moves along the outer periphery of the object E. FIG. 7A exemplifies a case in which the object E is divided into a plurality of layers in the direction Z in consideration of the size of the high sensitivity region G. FIG. 7B shows a path of the center of the high sensitivity region G at each layer and the high sensitivity region G at each measurement position. FIG. 7C shows, in an XZ plane, the path of the center of the high sensitivity region G and the position of the high sensitivity region G at each measurement position. Even in this case, photoacoustic waves are not received when a high sensitivity region exists in a region where the object does not exist. Therefore, it is possible to efficiently acquire received signals used in acquiring high-resolution object information about the interior of the object.

As described above, on the basis of coordinate information about a surface of an object, the photoacoustic apparatus according to the present embodiment determines a movement region in which the support member is moved so as to receive photoacoustic waves when a high sensitivity region exists at the position of the object. This makes it possible to preferentially receive photoacoustic waves generated from a region where the object exists. That is, it is possible to efficiently acquire a received signal for increasing the resolution of object information for the region where the object exists.

### Second Embodiment

In a second embodiment, an example in which a movement region of the support member 400 is set from coordinate information about a region whose object information is to be acquired (hereunder referred to as a “region of interest”) is described. According to the present embodiment, it is possible to preferentially receive photoacoustic waves generated at the region of interest. That is, it is possible to efficiently acquire a received signal for increasing the resolution of object information for the region of interest. It is possible to consider that the entire object corresponds to the region of interest in the first embodiment.

A method for acquiring object information for the interior of the region of interest by setting a movement region on the basis of the coordinate information about the region of interest using the photoacoustic apparatus shown in FIG. 1 is hereunder described.

First, the computing unit 710, serving as a region-of-interest setting unit, sets the region of interest, and acquires coordinate information about the region of interest.

For example, a user inputs information about the region of interest using the input unit 1000, and the input information is transmitted to the computer 700. Next, the computing unit 710 sets the region of interest on the basis of the input information about the region of interest, and acquires the coordinate information about the region of interest. More specifically, among images of the object displayed on the display 900, the user specifies a region that becomes the region of interest using the input unit 1000. This allows the region specified using the input unit 1000 to be transmitted to the computer 700 as the region of interest. Here, photoacoustic apparatuses, ultrasonic diagnostic apparatuses, and various image forming apparatuses, such as computerized tomography (CT) apparatuses and magnetic resonance imaging (MRI) apparatuses, are capable of acquiring an image of the object that is displayed on the display 900. The image of the object acquired using an image forming apparatus may be an image of the interior of the object.

However, an image forming apparatus may perform a measurement in a measurement state (such as the shape of the object) that differs from a state of measurement using the photoacoustic apparatus. In this case, it is desirable for the computing unit 710 to convert coordinates of the image of the object that is displayed on the display 900 into coordinates of an image that can be acquired by the photoacoustic apparatus according to the present embodiment, or it is desirable that the computing unit 710 convert the coordinate information about the region of interest that has been specified on the basis of the image acquired by the image forming apparatus into coordinate information about the image that can be acquired by the photoacoustic apparatus according to the present embodiment.

Alternatively, the computing unit 710 may extract a region of a portion to be observed from the image acquired by the image forming apparatus, and set this region as the region of interest. For example, it is possible for the computing unit 710 to determine that a region having high similarity with respect to the structure of the portion to be observed is the region of interest, to set this region as the region of interest and acquire coordinate information about this region. More specifically, when the object is a breast, it is possible to set the region of interest using data about typical structures of an upper inner portion of the breast (region A), a lower inner portion of the breast (region B), an upper outer portion of the breast (region C), a lower outer portion of the breast (region D), a lower portion of an areola (region E), and an axillary tail of the breast (region C′). First, using the input unit 1000, a user inputs information about a portion that the user wants to observe from these plurality of portions of the breast. Next, the computing unit 710 acquires information regarding similarity between input structural data about the portion of the breast and the image acquired by the image forming apparatus, so that a highly similar region can be set as the region of interest.

When, for example, a region where a tumor exists or a region where it is suspected that a tumor exists is previously known, these regions are repeatedly measured as time passes, so that comparative evaluations in terms of, for example, changes resulting from medication and changes with time are ordinarily performed. When a portion where such changes are subjected to the comparative evaluation is defined as the region of interest, the computing unit 710 can acquire information regarding the similarity between structural data about the portion subjected to the comparative evaluation previously acquired by the image forming apparatus and the image acquired by the image forming apparatus, and set the highly similar region as the region of interest. By setting the region of interest in this way, it is possible to increase reproducibility of the position when the same region of interest is repeatedly measured.

Next, the computing unit 710, serving as a movement region setting unit, sets the movement region of the support member 400 on the basis of the set coordinate information about the region of interest. At this time, the computing unit 710 sets the movement region in the directions X, Y, and Z of the support member 400 on the basis of the coordinate information about the region of interest so that a high sensitivity region G is formed at the inner side of the region of interest. The position and size of the high sensitivity region G are determined by the arrangement of the plurality of transducers 300. Accordingly, on the basis of the coordinate information about the region of interest and the information about the arrangement of the plurality of transducers 300 at the support member, the computing unit 710 can set the movement region so that the high sensitivity region G is formed at the inner side of the region of interest. The information about the size and position of the high sensitivity region G that are determined from the arrangement of the plurality of transducers 300 may be previously stored in the storage unit 720. In this case, the computing unit 710 may set the movement region on the basis of the information about the size and position of the high sensitivity region G read out from the storage unit 720 and the coordinate information about the region of interest.

It is desirable to set the movement region of the support member 400 so that measurements are performed when the center O of the high sensitivity region G at each measurement position is provided at the inner side of the region of interest. That is, in the present embodiment, it is desirable that the movement region be set so that measurements are performed when the region of interest exists at the center of curvature of the hemispherical support member 400 at each measurement position.

Further, it is desirable to set the movement region so that measurements are performed when the center of the high sensitivity region G corresponding to the outermost periphery of the movement region matches an outer edge of the region of interest.

As described above, the movement region in which the support member is moved for measuring at the high sensitivity region photoacoustic waves generated at the region of interest is determined on the basis of the set coordinate information about the region of interest. Therefore, it is possible to efficiently acquire with high sensitivity photoacoustic waves generated at the region of interest.

In third and fourth embodiments, exemplary methods for suitably moving the support member 400 in the set movement region are hereunder described. In the third and fourth embodiments, a case in which photoacoustic waves are received at equal time intervals by continuously moving the support member 400 and periodically applying light is described. However, the timing of receiving photoacoustic waves can be set as appropriate by changing the movement speed of the support member 400 and light emission timing. For convenience, in the figures used in the third and fourth embodiments, the high sensitivity region G at each measurement position is not shown.

### Third Embodiment

A photoacoustic apparatus according to the third embodiment is hereunder described using the photoacoustic apparatus according to the first embodiment shown in FIG. 1.

In the third embodiment, a scanner 500 causes the support member 400 to undergo circular movement. The term “circular movement” in the present embodiment refers to a curvilinear movement similar to a circular movement and an elliptical movement.

When the movement region having a curved surface, such as a hemispherical surface or a conical surface, is set and the support member 400 is moved so that a plurality of high sensitivity regions exist on the curved surface, circular movement is more suitable than the linear movement described in the first embodiment. That is, when an object, such as a breast, whose shape is similar to a conical shape or a hemispherical shape is to be measured, if a movement region is set so that the plurality of high sensitivity regions are provided along an external form of the object, it is desirable for the support member 400 to undergo circular movement than linear movement. When the photoacoustic apparatus is formed so that the input unit 1000 is capable of inputting information about a region of interest having a curved surface, it is similarly desirable for the support member 400 to undergo circular movement than linear movement. This is because, when, in linearly moving the support member 400, an attempt is made to perform measurements so that the high sensitivity regions exist on the curved surface, the measurements need to be performed by changing directions over and over again, as a result of which measurement time becomes long. The computing unit 710 is capable of determining whether or not to cause the support member 400 to undergo linear movement or circular movement, on the basis of the size of the high sensitivity regions and the curvature of the outer periphery of the movement region. However, even if the movement region is one including a curved surface, when the high sensitivity regions at the measurement positions include the entire movement region in one linear movement, the scanner 500 may linearly move the support member 400.

The acoustic matching material 800 with which the container of the support member 400 is filled is subjected to inertial force due to the movement of the support member 400. When the support member 400 undergoes linear movement, if the direction is repeatedly changed, the acoustic matching material 800 may become foamy as a result of a change in a liquid level due to the inertial force. Therefore, a location between the object E and the plurality of transducers 300 may not be filled up with the acoustic matching material 800. In contrast, when the support member 400 undergoes circular movement, the acoustic matching material 800 is subjected to a force in an outer peripheral direction of the circular movement at all times. Therefore, compared to a movement pattern formed by the linear movement in which the direction is repeatedly changed, the circular movement makes it possible to gradually change the liquid level. Therefore, acoustic matching between the object E and the plurality of transducers 300 is facilitated.

The rotational axis of the circular movement of the support member 400 may be changed in accordance with the movement region. That is, it is desirable that, in accordance of the movement region, the computing unit 710 set a movement path so That the rotational axis of the circular movement of the support member 400 passes through the center of the movement region.

An example of a specific circular movement of the support member 400 is described below.

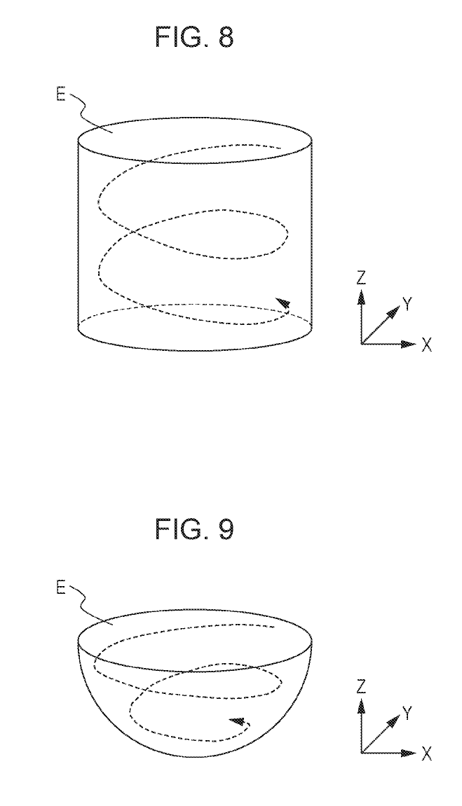

An example in which the scanner 500 circularly moves the support member 400 when a movement path is set so that a plurality of high sensitivity regions exist along an external form of an object E having a cylindrical shape shown in FIG. 8 is described. The cross sections of the cylinder in a height direction (direction Z) are the same. In this case, it is desirable that the scanner 500 cause the support member 400 to undergo a helical movement in which, while the support member 400 moves in the height direction of the cylinder, the support member 400 undergoes a circular movement at a same turning radius with an axis in the direction Z passing through the center of the cylinder being defined as the rotational axis. The dotted lines in FIG. 8 indicate a path of the center of the high sensitivity region G as the support member 400 moves. On the basis of coordinate information about a surface of the object. E and the size of the high sensitivity region G, the computing unit 710 computes the movement path of the high sensitivity region G shown in FIG. 8 and sets the movement region of the support member 400 that is suitable for the cylindrical object.

In measuring an object whose cross sections are the same in the height direction, it is possible to acquire a received signal with a small movement distance by causing the support member 400 to undergo a helical movement that has been set on the basis of coordinate information about a surface of the object. Unlike the case in which the support member 400 is linearly moved and the directions are changed, it is possible to continuously move the support member 400 with respect to the cylindrical object E as long as the movement is a helical movement. This makes it possible to further reduce the time required for acquiring a received signal.

It is possible to helically move the support member 400 with respect to movement, regions other than a cylindrical movement region. For example, it is possible to helically move the support member 400 with respect to a movement region having a shape that is similar to, for example, the shape of a prism whose cross sectional areas in the height direction are the same.

Next, an example of a circular movement that is suitable for a case in which the support member 400 is moved so that a plurality of high sensitivity regions exist in a movement region having a shape whose cross sections change in the height direction is described.

The cross section in the height direction (direction Z) of a hemispherical object E shown in FIG. 9 changes. In this case, considering the size of the high sensitivity regions G, it is desirable to cause the support member 400 to undergo a three-dimensional spiral movement in which the support member 400 undergoes a circular movement with varying turning radii while moving in a height direction of a hemisphere. The dotted lines in FIG. 9 indicate a path of the centers of the high sensitivity regions G as the support member 400 moves.

Here, when the support member 400 is caused to undergo the spiral movement at the same speed, it is desirable that the support member 400 start moving from an outer periphery having a large radius and that the radius of the circular movement be reduced as the support member 400 moves. Such a movement makes it possible to efficiently receive with high sensitivity photoacoustic waves generated in the interior of the object E. In addition, it is possible to gradually change a force that the acoustic matching material 800 receives in an outer peripheral direction. As mentioned above, when the force applied to the acoustic matching material 800 gradually changes, a change in the wave surface of the acoustic matching material 800 is small, so that acoustic matching is facilitated.

It is possible to cause the support member 400 to undergo a spiral movement in movement regions other than hemispherical movement regions. For example, it is possible to cause the support member 400 to undergo a spiral movement even in a movement region having a shape that is similar to, for example, a cone or a pyramid whose cross-sectional area changes in a height direction.

In order to move the support member 400 only in one plane depending upon the shape of an object E and the size of a high sensitivity region G, the support member 400 may undergo a two-dimensional spiral movement.

### Fourth Embodiment

A photoacoustic apparatus according to the fourth embodiment is hereunder described using the photoacoustic apparatus according to the first embodiment shown in FIG. 1.

In the fourth embodiment, a case in which a scanner 500 causes a support member 400 to undergo a combination of a plurality of circular movements is described. Even in the present embodiment, the term “circular movement” refers to a curvilinear movement similar to a circular movement and an elliptical movement.

In the helical movement and the spiral movement described in the third embodiment, a region where a high sensitivity region does not exist becomes large when the high sensitivity region G becomes rather small with respect to the movement region. Therefore, it becomes difficult to receive with high sensitivity received signals of photoacoustic waves generated in the region where the high sensitivity region does not exist. As a result, an irregularity occurs in the resolution of an obtained piece of object information. For example, when the center of the high sensitivity region G is moved along an outer periphery of an object, the region where the high sensitivity region does not exist at an inner side of the outer periphery may become large.

Therefore, in the present embodiment, the support member 400 is caused to undergo a combination of a plurality of circular movements so that the high sensitivity region G exists in a wide-range region at the inner side of the movement region. Consequently, compared to the case in which the support member 400 is caused to undergo one circular movement, it is possible to receive with high sensitivity photoacoustic waves generated in a wide range within the movement region. As a result, the irregularity occurring in the resolution of an obtained piece of object information is reduced.

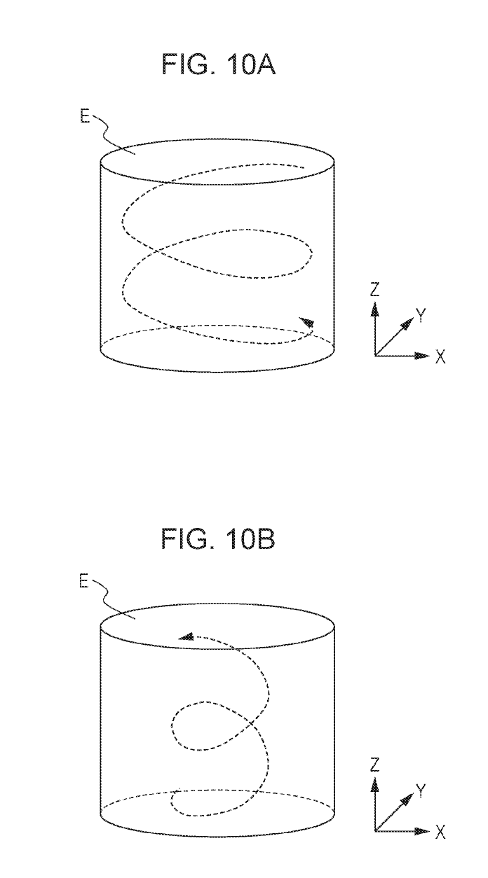

FIGS. 10A and 10B illustrate a case in which the support member 400 undergoes a plurality of helical movements at a cylindrical object E. The dotted lines in FIGS. 10A and 10B indicate a path of the center of a high sensitivity region G as the support member 400 moves.

First, the scanner 500 causes the support member 400 to undergo a first helical movement so that a plurality of high sensitivity regions exist at an outer periphery of an object (FIG. 10A). As mentioned above, if the support member 400 is only moved as mentioned above, an irregularity may occur in the resolution of object information.

Next, in order to reduce the irregularity in the resolution of the interior of the object, the scanner 500 causes the support member 400 to undergo a second helical movement whose turning radius differs from the turning radius of the first helical movement (FIG. 10B). This makes it possible to more the support member 400 so that the high sensitivity regions also exist in the interior of the cylindrical object, and the irregularity in the resolution of the object information is reduced.

As shown in FIGS. 10A and 10B, it is possible to smoothly switch between the first helical movement and the second helical movement by starting the first helical movement from a top surface of the cylinder and switching to the second helical movement when the first helical movement causes the support member 400 to be at a bottom surface of the cylinder. That is, the first helical movement and the second helical movement can be continuous. This makes it possible to reduce measurement time, and to reduce changes in the wave surface of the acoustic matching material 800.

It is possible to cause the support member 400 to undergo a plurality of helical movements even in a movement region having a shape that is similar to a prism whose cross-section area in a height direction is the same.

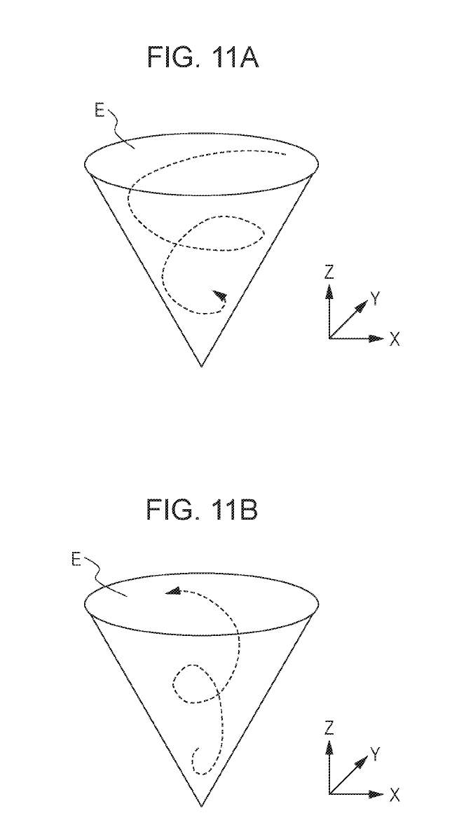

FIGS. 11A and 11B illustrate an example in which the support member 400 is caused to undergo a plurality of three-dimensional spiral movements at a conical object E whose cross section changes in a height direction (direction Z) of a movement region. In order to uniformly measure a region in a cone at a plurality of high sensitivity regions G, first, as shown in FIG. 11A, the position of the support member 400 is moved by a first spiral movement. Next, as shown in FIG. 11B, the position of the support member 400 is moved by a second spiral movement in a region differing from that where the first spiral movement is performed. The dotted lines in FIG. 11 indicate a path of the center of the high sensitivity regions G as the support member 400 moves. In the illustrated example, a region at the side of an outer periphery of an interior of the cone is measured on the basis of the first spiral movement, and a region at the side of the center of the interior of the cone is measured on the basis of the second spiral movement. As shown in FIGS. 11A and 11B, it is possible to continuously smoothly switch between the first spiral movement and the second spiral movement by starting the first spiral movement from a bottom portion of the cone and switching to the second spiral movement at the vertex of the cone. In this way, on the basis of coordinate information about a surface of the object and the size of the high sensitivity regions G, the computing unit 710 computes a movement path shown in FIGS. 11A and 11B and sets the movement region in which the support member 400 is moved suitable for the conical object E.

By causing the support member 400 to undergo a plurality of spiral movements in this way, compared to the case in which the support member 400 is caused to undergo one spiral movement, it is possible to receive with high sensitivity photoacoustic waves generated from a wide range in the interior of the object.

It is possible to cause the support member 400 to undergo a plurality of spiral movements in movement regions other in conical movement regions.

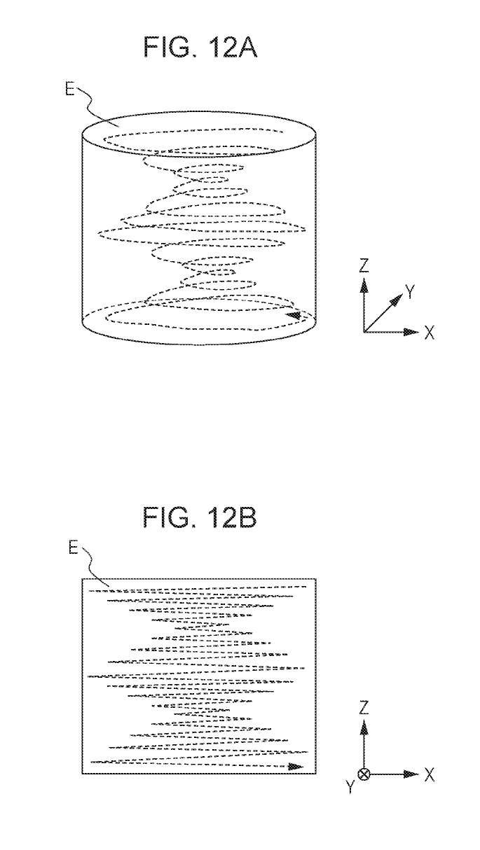

FIGS. 12A and 12B show a case in which a spiral movement whose turning radius is changed from “large to small” towards a direction Z and a spiral movement whose turning radius is changed from “small to large” towards the direction Z are repeated a plurality of times to move the support member 400 is described. The dotted lines in FIGS. 12A and 122 indicate a path of the center of the high sensitivity region G as the support member 400 moves.

By causing the support member 400 to undergo such spiral movements, compared to the case in which the support member 400 is caused to undergo one spiral movement, it is possible to receive with high sensitivity photoacoustic waves generated from a wide range in the interior of an object. For example, when the turning radius of the spiral movement is large, a high sensitivity region may not exist near the center of the spiral movement. Therefore, it is desirable to move the support member 400 so that the high sensitivity region when the turning radius of the spiral movement is small overlaps the vicinity of the center of the spiral movement when the turning radius is large. This makes it possible to receive with high sensitivity photoacoustic waves generated in the vicinity of the center of the spiral movement when the turning radius is large.

By changing the turning radius of the spiral movement from “small to large” after changing the turning radius of the spiral movement from “large to small”, it is possible to continuously smoothly switch between the plurality of spiral movements. This makes it possible to reduce movement time of the support member 400 and measurement time.

By setting as appropriate the turning radius of each spiral movement on the basis of coordinate information about a surface an object E, it is possible to also apply each spiral movement to movement regions other than hemispherical movement regions.

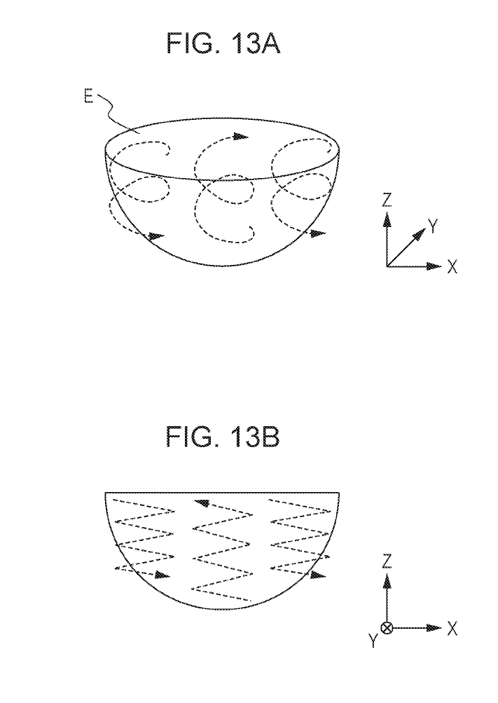

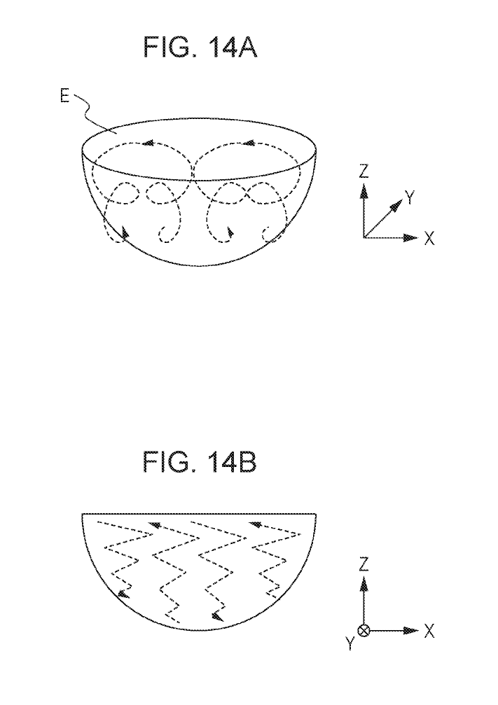

FIGS. 13A and 13B show a case in which the support member 400 is caused to undergo a plurality of helical movements whose turning radius is smaller than the radius of an outer periphery of a movement region. The dotted lines in FIGS. 13A and 13B indicate a path of the center of a high sensitivity region G as the support member 400 moves. FIGS. 14A and 14B show a case in which the support member 400 is caused to undergo a plurality of spiral movements whose turning radius is smaller than the radius of an outer periphery of a movement region. The dotted lines in FIGS. 14A and 14B each indicate a path of the center of a high sensitivity region G as the support member 400 moves. In the cases shown in FIGS. 13A to 14B, a hemispherical movement region that is suitable for a hemispherical object E is assumed.

In FIGS. 13A to 14B, the support member 400 is caused to undergo a combination of a plurality of helical movements or spiral movements whose turning radius is smaller than the radius of the outer periphery of the movement, region. According to these cases, compared to the case in which the support member 400 is caused to undergo one helical movement or one spiral movement, it is possible to receive with high sensitivity photoacoustic waves generated from a wide range in the interior of the object.

It is possible to cause the support member 400 to undergo a combination of a plurality of helical movements or spiral movements whose turning radius is small for movement regions other than hemispherical movement regions.

In FIGS. 14A and 14B, it is possible to change the rotational axis for each depth of the helical movement. This makes it possible to also apply the present invention to complicated movement regions by small movement amounts.

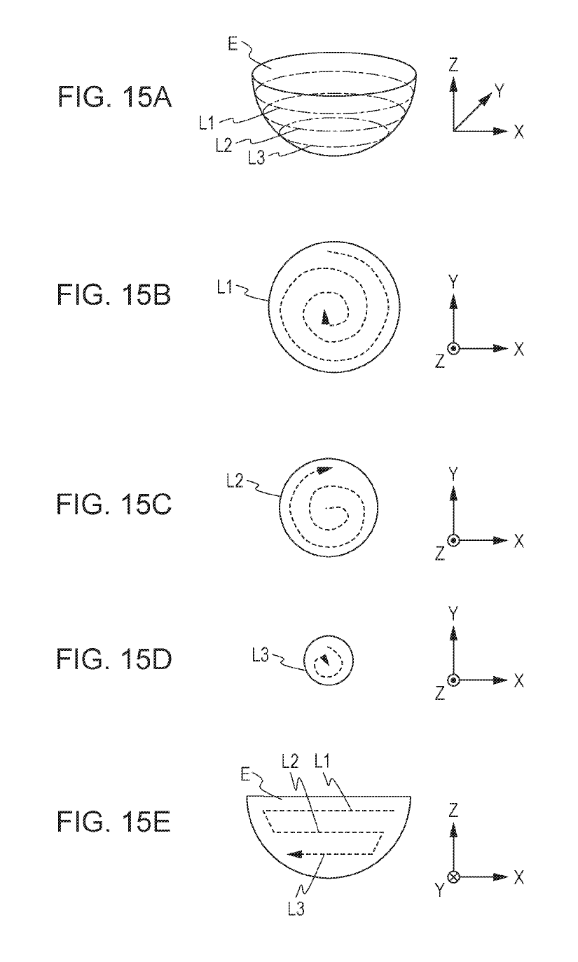

FIGS. 15A to 15E show a case in which the support member 400 is caused to undergo spiral movements having different outermost diameters in corresponding planes (XY planes) on the basis of coordinate information about a surface of an object E. The dotted lines in FIGS. 15A to 15E each indicate a path of the center of a high sensitivity region G as the support member 400 moves. A hemispherical movement region suitable for the hemispherical object E is assumed.

As shown in FIG. 15A, the hemispherical movement region suitable for the object E is divided into three layers, that is, layers L1, L2, and L3.

FIG. 15B shows a path of the center of the high sensitivity region G at the layer L1. In the layer L1, the support member 400 is caused to undergo three spiral movements towards an inner side of the movement region from an outer side of the movement region while the turning radius of a two-dimensional spiral movement is changed in a radial direction.

FIG. 15C shows a path of the center of the high sensitivity region G at the layer L2. In the layer L2, the support member 400 is caused to undergo two spiral movements from the inner side of the movement region towards the outer side of the movement region while the turning radius of the two-dimensional spiral movement is changed in the radial direction. In this way, it is possible to smoothly start the spiral movement at each layer by starting the two-dimensional spiral movement from the inner side in the layer L2 after the two-dimensional spiral movement up to the inner side in the layer L1. This reduces movement time and measurement time.

FIG. 15D shows a path of the center of the high sensitivity region G at the layer L3. In the layer L3, the support member 400 is caused to undergo one spiral movement towards the inner side of the movement region from the outer side of the movement region while the turning radius of the two-dimensional spiral movement is changed in the radial direction.

By causing the support member 400 to undergo two-dimensional spiral movement in each plane, compared to the case in which the support member 400 is caused to undergo two-dimensional spiral movement in one plane, it is possible to receive with high sensitivity photoacoustic waves generated from a wide range in the interior of the object.

It is also possible to apply the plurality of two-dimensional spiral movements to movement regions other than hemispherical movement regions.

By moving the support member 400 according to the present embodiment as described above, compared to the case in which the support member is caused to undergo one circular movement in the movement region, it is possible to receive with high sensitivity photoacoustic waves generated from a wide range in the interior of the object. As a result, irregularity in the resolution of the acquired object information is reduced.

Since the scanner 500 circularly moves the support member 400, the acoustic matching material 800 is subjected to a force in an outer peripheral direction of the circular movement at all times. Therefore, the change in shape of the acoustic matching material 800 is gradual, so that acoustic matching between the object and the transducers 300 is facilitated. In addition, when the support member 400 is caused to continuously undergo a plurality of circular movements, the force in the outer peripheral direction that is applied to the acoustic matching material 800 can be further gradually changed. Therefore, acoustic matching between the object and the transducers 300 is further facilitated.

### Fifth Embodiment

In a fifth embodiment, an example, in which at least some of a plurality of transducers 300 arranged at a support member 400 are used as an imaging element 610 is described.

The transducers 300 are arranged so as to face the center of a high sensitivity region G. This limits the effective critical angle of the transducers 300, so that it is possible to more efficiently receive photoacoustic waves of the high sensitivity region G.

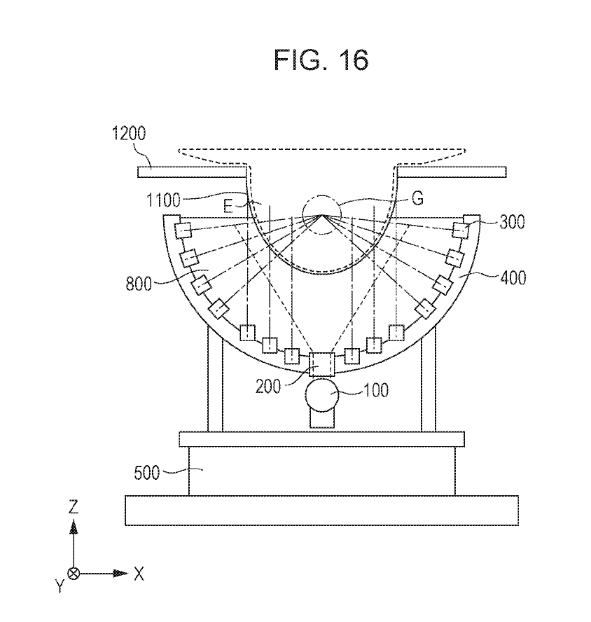

Therefore, in the present embodiment, it is possible to transmit acoustic waves from some of the plurality of transducers 300 arranged as shown in FIG. 16 and receive reflected waves (echoes) of the transmitted acoustic waves by the at least some of the transducers 300. A computing unit 710 is capable of acquiring a B-mode image from a received signal of the echo acquired in this way. As mentioned above, on the basis of the B-mode image acquired in this way, it is possible for the computing unit 710 to acquire coordinate information about a surface of an object E by image processing. In addition, it is possible to acquire the coordinate information about the surface of the object E by causing a display 900 to display the B-mode image and a user to specify an external shape of the object E in the B-mode image using an input unit 1000. This structure makes it possible to acquire the coordinate information about the surface of the object without adding hardware.

When some of the plurality of transducers 300 are used as the imaging element 610, since a receiving direction of the transducers 300 is towards the center of the high sensitivity region G, the receiving sensitivity G with respect to an echo that is generated at a region other than the high sensitivity region is low. Therefore, the quality of the B-mode image at regions other than the high sensitivity region G is reduced. Consequently, it is difficult to precisely acquire coordinate information about the surface of the entire object E on the basis of the B-mode image.

Therefore, as shown in FIG. 16, some of the plurality of transducers 300 may be arranged so as to face a region other than the high sensitivity region G instead of the center of the high sensitivity region G. This arrangement makes it possible to precisely acquire the coordinate information about the surface of the entire object E. In particular, when it is assumed that the support member 400 is larger than the object E (for example, when the support member 400 is larger than a shape maintaining unit), arranging some of the plurality of transducers 300 so as to face a negative side of a Z axis makes it easier to acquire the coordinate information about the surface of the entire object E.

In the present embodiment, as shown in FIG. 16, the transducers 300 existing along the Z axis of a hole into which the breast E, which is an object, is inserted are arranged so as to face the negative side of the Z axis. In FIG. 16, among the plurality of transducers 300, only the transducers in a certain X-Z plane are shown as facing the negative side of the Z axis, all of the transducers existing along the Z axis of the hole into which the breast E is inserted actually face the negative side of the Z axis. These transducers are used to acquire a B-mode image.

However, since, typically, the sound speed in a shape maintaining unit 1100 and the sound speed in an acoustic matching material 800 differ from each other, acoustic waves transmitted from the transducers 300 are refracted at an interface between the shape maintaining unit 1100 and the acoustic matching material 800.

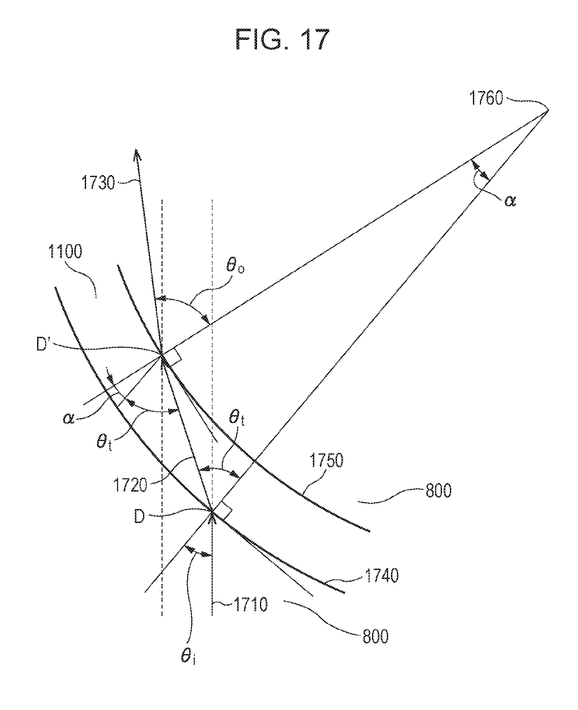

FIG. 17 illustrates details of refraction of acoustic waves between the shape maintaining unit 1100 and the acoustic matching material 800. In the present embodiment, a case in which the sound speed in the shape maintaining unit 1100 is higher than the sound speed in the acoustic matching material 800 is described.

An acoustic wave 1710 that is incident upon a point D of an outer boundary surface 1740 of the shape maintaining unit 1100 at an angle θᵢ is refracted at an angle θₜ to an inner portion of the shape maintaining unit 1100. Next, an acoustic wave 1720 that is incident upon a point D′ an inner boundary surface 1750 of the shape maintaining unit 1100 at an angle (θₜ+α) is refracted at an angle θₒ towards the inner side of the shape maintaining unit 1100 (upper side in FIG. 17). Next, a refracted acoustic wave 1730 propagates through the interior of the acoustic matching material 800. An angle that is formed by a straight line connecting the point D and a curvature center 1760 and a straight line connecting the point D′ and the curvature center 1760 is α.

These relationships are represented by Formulas (2) and (3) by Snell's law:

$$\begin{matrix}[ Math . 2 ] & \\ \frac{sin {θ}_{i}}{sin {θ}_{t}} = \frac{{c}_{i}}{{c}_{t}} & ( 2 ) \\ [ Math . 3 ] & \\ \frac{sin ( {θ}_{t} + α )}{sin {θ}_{o}} = \frac{{c}_{t}}{{c}_{i}} & ( 3 )\end{matrix}$$

From Formulas (2) and (3), Formula (4) can be derived:

$$\begin{matrix}[ Math . 4 ] & \\ \frac{sin {θ}_{o}}{sin {θ}_{t}} = \frac{sin ( {θ}_{t} + α )}{sin {θ}_{t}} & ( 4 )\end{matrix}$$

In FIG. 17, since 0<α<90 degrees and 0<(α+θₜ)<90 degrees, Formula (4) has the following relationship expressed by Formula (5):

$$\begin{matrix}[ Math . 5 ] & \\ \frac{sin {θ}_{o}}{sin {θ}_{i}} = \frac{sin ( {θ}_{t} + α )}{sin {θ}_{t}} > 1 & ( 5 )\end{matrix}$$

Therefore, it is possible to obtain the relationship expressed by Formula (6):

[Math. 6]

sin θₒ>sin θᵢ (6)

That is, at the inner side of the shape maintaining unit 1100 (upper side in FIG. 17), an acoustic wave incident from an outer side of the shape maintaining unit 1100 (lower side in FIG. 17) is refracted at an angle that is greater than an incidence angle. Therefore, when a movement region is set on the basis of coordinate information about a surface of an object acquired from a received signal acquired without considering this refraction, a region that is larger than the size of the actual object is set as the movement region.

Therefore, when, as shown in FIG. 17, the receiving direction is towards the negative side of the Z axis, the computing unit 710 acquires a B-mode image on the basis of Snell's law in Formulas (2) and (3). According to this, the computing unit 710 is capable of acquiring a B-mode image that approximates to the shape of the actual object. Further, by acquiring coordinate information about the surface of the object by image processing on the basis of the B-mode image that approximates to the shape of the actual object, it is possible to acquire the coordinate information about the surface of the object that approximates to the shape of the actual object. On the basis of the coordinate information about the surface of the object acquired in this way, the computing unit 710 is capable of setting a movement region that is in accordance with the shape that approximates to that of the actual object.

Consider a case in which coordinate information about a surface of an object from a B-mode image acquired without considering the refraction of acoustic waves. In this case, by image processing based on Snell's law, the computing unit 710 can acquire coordinate information about a surface of an object with a region being smaller than the object indicated by the B mode being set as an object region. According to this, even if a B-mode image of an object that is larger than the form of an actual object is acquired due to refraction, it is possible to set a movement region that is in accordance with the shape of the actual object considering the refraction.

In the present embodiment, the case in which the sound speed in the shape maintaining unit 1100 is higher than the sound speed in the acoustic matching material 800 is described. The present embodiment is also applicable to a case in which the sound speed in the shape maintaining unit 1100 is lower than the sound speed in the acoustic matching material 800. That is, when the sound speed in the shape maintaining unit 1100 is lower than the sound speed in the acoustic matching material 800, it is possible to perform corrections considering refraction that are similar to those described above on the basis of Snell's law.

Further, in satisfying Formula (7) or (8), total reflection of transmitted acoustic waves at the outer boundary surface 1740 or at the inner boundary surface 1750 of the shape maintaining unit 1100 becomes a problem:

$$\begin{matrix}[ Math . 7 ] & \\ sin {θ}_{i} ≧ \frac{{c}_{i}}{{c}_{t}} & ( 7 ) \\ [ Math . 8 ] & \\ sin ( {θ}_{t} + α ) ≧ \frac{{c}_{t}}{{c}_{i}} & ( 8 )\end{matrix}$$

Therefore, it is desirable to arrange the transducers 300 so that the acoustic waves that are transmitted from the transducers 300 that transmit and receive the acoustic waves not be totally reflected at the outer boundary surface 1740 or the inner boundary surface 1750 of the shape maintaining unit 1100. According to this arrangement, it is possible for the transmitted acoustic waves to reach a surface of an object without being totally reflected. Therefore, it is possible to acquire a B-mode image including the object.

Further, in order to reduce total reflection at the outer boundary surface 1740 of the shape maintaining unit 1100, it is desirable that the receiving direction (directivity axis) of each transducer 300 that transmits and receives acoustic waves be arranged in a direction normal to a curved surface of the shape maintaining unit 1100. In this case, since the refraction at the shape maintaining unit 1100 is reduced, even if the refraction is not considered, the computing unit 710 is capable of acquiring a B-mode image that approximates to the shape of an actual object. Therefore, a movement region that is in accordance with the shape of the actual object can be set on the basis of the obtained B-mode image without performing an additional processing operation.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiments of the present invention, and by a method performed by the computer of the system or apparatus by, for example, reading out and executing the computer executable instructions from the storage medium to perform the functions of one or more of the above-described embodiments. The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. provisional application No. 61/873,542, filed Sep. 4, 2013, U.S. provisional application No. 61/898,025, filed Oct. 31, 2013, which are hereby incorporated by reference herein in their entirety.

1. A photoacoustic apparatus comprising:
a light source;
a plurality of transducers configured to receive acoustic waves and output electric signals, the acoustic waves being generated when an object is irradiated with light generated from the light source;
a support member configured to support the plurality of transducers such that directivity axes of the plurality of transducers gather;
a movement region setting unit configured to set a movement region of the support member;
a moving unit configured to move the support member in the movement region such that relative position between the object and the support member changes; and
an information acquiring unit configured to acquire object information on the basis of the electric signals,
wherein the light source emits the light when the support member is positioned in the movement region, and
wherein the movement region setting unit acquires coordinate information about a surface of the object and determines the movement region on the basis of the coordinate information about the surface of the object.

2. The photoacoustic apparatus according to claim 1, wherein the light source generates the light at a plurality of timings, and wherein the movement region setting unit sets the movement region on the basis of the coordinate information about the surface of the object so that at least part of a high sensitivity region defined by the plurality of transducers overlaps the object at the plurality of timings.

3. The photoacoustic apparatus according to either claim 1, wherein the light source generates the light at a plurality of timings, and wherein the movement region setting unit sets the movement region so that a high sensitivity region defined by the plurality of transducers is positioned in the object at the plurality of timings.

4. The photoacoustic apparatus according to of claim 1, wherein the light source generates the light at a plurality of timings, and wherein the movement region setting unit sets the movement region so that a position where the directivity axes of the plurality of transducers gather is positioned in the object at the plurality of timings.

5. The photoacoustic apparatus according to claim 1 4, further comprising an imaging element, wherein the movement region setting unit acquires the coordinate information about the surface of the object on the basis of signals output from the imaging element.

6. The photoacoustic apparatus according to claim 5, wherein the imaging element is at least one of the plurality of transducers, and wherein the at least one of the plurality of transducers transmits the acoustic waves and receives reflected waves of the acoustic waves to output the signals.

7. The photoacoustic apparatus according to claim 6, further comprising a mount unit configured to mount on a shape maintaining unit for maintaining a shape of the object, wherein the at least one of the plurality of transducers is disposed at the support member so that the directivity axis of the at least one of the plurality of transducers is oriented in a direction that is normal to a surface of the shape maintaining unit.

8. The photoacoustic apparatus according to claim 1 4, further comprising a mount unit configured to mount on a shape maintaining unit for maintaining a shape of the object, and a storage unit configured to store coordinate information about a surface of the shape maintaining unit, wherein the movement region setting unit acquires, as the coordinate information about the surface of the object, the coordinate information about the surface of the shape maintaining unit stored in the storage unit.

9. The photoacoustic apparatus according to claim 1 4, further comprising a mount unit configured to mount on or remove a plurality of shape maintaining units for maintaining a plurality of shapes of the object, and a storage unit configured to store coordinate information about a surface of each of the plurality of shape maintaining units, wherein the movement region setting unit reads out coordinate information about the surface of the shape maintaining unit mounted on the mount unit from the coordinate information about the surface of each of the plurality of shape maintaining units stored in the storage unit, and acquires the read out coordinate information as the coordinate information about the surface of the object.

10. The photoacoustic apparatus according to claim 9, further comprising an input unit configured to allow a user to input information on the type of shape maintaining unit mounted on the mount unit from the types of the plurality of shape maintaining units, wherein, on the basis of an output from the input unit, the movement region setting unit reads out the coordinate information about the surface of the shape maintaining unit mounted on the mount unit from the coordinate information about the surface of each of the plurality of shape maintaining units stored in the storage unit, and acquires the read out coordinate information as the coordinate information about the surface of the object.

11. The photoacoustic apparatus according to claim 9, further comprising a detecting unit configured to detect the type of shape maintaining unit mounted on the mount unit, wherein, on the basis of an output from the detecting unit, the movement region setting unit reads out the coordinate information about the surface of the shape maintaining unit mounted on the mount unit from the coordinate information about the surface of each of the plurality of shape maintaining units stored in the storage unit, and acquires the read out information as the coordinate information about the surface of the object.

12. The photoacoustic apparatus according to claim 1, wherein the movement region setting unit sets the movement region on the basis of the coordinate information about the surface of the object and an arrangement of the plurality of transducers at the support member.

13. A photoacoustic apparatus comprising:
a light source;
a plurality of transducers configured to receive acoustic waves and output electric signals, the acoustic waves being generated when an object is irradiated with light generated from the light source;
a support member configured to support the plurality of transducers such that directivity axes of the plurality of transducers gather;
a movement region setting unit configured to set a movement region of the support member;
a moving unit configured to move the support member in the movement region such that relative position between the object and the support member changes;
an input unit configured to allow a user to input information about a region of interest;
a region-of-interest setting unit configured to set the region of interest on the basis of an output from the input unit; and
an information acquiring unit configured to acquire object information about the region of interest on the basis of the electric signals,
wherein the light source emits the light when the support member is positioned in the movement region, and
wherein the movement region setting unit determines the movement region on the basis of coordinate information about the region of interest.

14. A photoacoustic apparatus comprising:
a light source;
a plurality of transducers configured to receive acoustic waves and output electric signals, the acoustic waves being generated when an object is irradiated with light generated from the light source;
a support member configured to support the plurality of transducers such that directivity axes of the plurality of transducers gather;
a movement region setting unit configured to set a movement region of the support member;
a moving unit configured to move the support member in the movement region such that relative position between the object and the support member changes; and
an information acquiring unit configured to acquire object information on the basis of the electric signals,
wherein the light source emits the light when the support member is positioned in the movement region, and
wherein the movement region setting unit is capable of changing the movement region.

15. The photoacoustic apparatus according to claim 1, wherein the light source generates the light when the support member is positioned at each of a plurality of positions in the movement region, and wherein the plurality of transducers receive the acoustic waves and output the electric signals, the acoustic waves being generated as a result of irradiating the object with the light when the support member is positioned at each of the plurality of positions in the movement region.

16. The photoacoustic apparatus according to claim 1, wherein the moving unit causes the support member to undergo a two-dimensional circular movement in the movement region.

17. The photoacoustic apparatus according to claim 1, wherein the moving unit causes the support member to undergo a two-dimensional circular movement in each of a plurality of planes in the movement region.

18. The photoacoustic apparatus according to either claim 16, wherein the two-dimensional circular movement is a two-dimensional spiral movement.

19. The photoacoustic apparatus according to claim 1, wherein the moving unit causes the support member to undergo a three-dimensional circular movement in the movement region.

20. The photoacoustic apparatus according to claim 1, wherein the moving unit causes the support member to undergo a plurality of three-dimensional circular movements in the movement region.

21. A photoacoustic apparatus comprising:
a light source;
a plurality of transducers configured to receive acoustic waves and output electric signals, the acoustic waves being generated when an object is irradiated with light generated from the light source;
a support member configured to support the plurality of transducers such that directivity axes of the plurality of transducers gather;
a moving unit configured to cause the support member to undergo continuous circular movement; and
an information acquiring unit configured to acquire object information on the basis of the electric signals,
wherein the light source emits the light while the moving unit causes the support member to undergo the continuous circular movement.
